(19) United States
(12) Patent Application Publication
(10) Pub. No.: US 2006/0032495 A1
(43) Pub. Date: Feb. 16, 2006

(54) UNIVERSAL FASTENER FOR ASTHMA INHALER

(76) Inventor: Jennifer Fernandez, Plantation, FL (US)

Correspondence Address: MALIN HALEY AND DIMAGGIO, PA, 1936 S ANDREWS AVENUE, FORT LAUDERDALE, FL, 33316, US

(21) Appl. No.: 10/710,898

(22) Filed: Aug. 11, 2004

(51) Int. Cl. A61M 11/00; A61M 15/00; B65D 83/06

(52) U.S. Cl. 128/200.23; 128/203.15

(57) ABSTRACT

A device for securely attaching a medical inhaler that dispenses medication required by people who have asthma or other respiratory diseases that can be removably attached to the inhaler and includes the use of a spring loaded clip with jaws that will firmly attach to a garment and not be released.

### BACKGROUND OF INVENTION

1. Field of the Invention

This invention relates to an inhaler holder and securing device for securely attaching an inhaler to the garment or person of a user so that the inhaler is always available for instant use and, specifically, to an improved inhaler holder and fastener that allows the inhaler holder and inhaler to be securely fastened especially to garments of the user.

2. Description of Related Art

Individuals who suffer from asthma or other respiratory diseases must often have a medical inhaler close by. There are times when having the inhaler available during an asthma attack can mean the difference between life and death. The prior art shows a variety of holsters and pouches that are used to retain or secure an inhaler. U.S. Pat. No. 5,855,307 issued Jan. 5, 1999 shows an inhaler holster that can be used with a wrist band for securing the inhaler to your wrist. U.S. Pat. No. 6,360,929 issued Mar. 26, 2002 shows a medicinal inhaler that uses a fastener that attaches to a belt. There are many environments when the types of fasteners shown in the prior art are not effective. One example would be when wearing a bathing suit or garment without belts. The present invention overcomes these problems by providing a foolproof, universal inhaler fastener that can attach to any garment.

### SUMMARY OF INVENTION

A device for attaching an inhaler to a garment regardless of the garment configuration comprising a flexible, cylindrical housing that can be opened that includes hook and pile fasteners, the housing being sized to receive a conventional asthma inhaler. An alligator clip is tethered to the housing by a flexible cord that may include a D ring that attaches the alligator clip to the flexible cord. The body of the flexible, cylindrical housing may be made of plastic or fabric, either man-made or natural. One end of the cylindrical, flexible housing may be enclosed for securing the base of the inhaler within the cylindrical housing when the hook and pile fasteners have been closed together. The strap may be of a resilient plastic or fabric material that forms a strap that has closed connecting loops at each end.

The purpose of the flexible, cylindrical housing is to encase or encompass a standard, conventional inhaler. The inhaler is a container that has a mechanism for dispensing a medicant that is inhaled into the lungs. The inhaler itself has usually a rigid body that allows a portion to be used in the mouth of the user and a removable container that contains the medication that upon depression squirts medicant in through the mouth opening portion for the user. This rigid plastic housing is enclosed in applicant's invention with a flexible, cylindrical encasement or housing that is made of plastic and may use hook and pile fasteners along two edges to adjust to different size inhalers. Permanently attached along one edge of the flexible housing is a metal D ring and a circular passage to hold the D ring to the flexible housing. A strap that is tethered is also connected to the D ring. Finally, an alligator clip or yet another garment clip may be attached to the tether strap.

To use the present invention, the user would purchase an inhaler and wrap the invention and flexible housing around the inhaler body, securing the encasement firmly to the inhaler. At that point, the device is ready for use. By using an alligator clip or a garment clip with jaws, the device can be attached to virtually any part of a garment including a bathing suit or even underwear.

It is paramount that for an inhaler to be properly secured that the inhaler is firmly attached to the garment so that the inhaler cannot fall off accidentally or become removed. It is believed by using a strong alligator clip, the inhaler is securely fastened.

In an alternate embodiment, the flexible housing or encasement could be made in a rectangular strip sized in length to fit around the diameter of an inhaler and include an adhesive backing and a removable protective adhesive strip so that the flexible housing could be adhesively attached permanently to the inhaler body. The adhesive resilient housing would also include the D ring permanently attached thereto, a tether strap and the alligator clip attached thereto. This could be marketed in the stores so that each time an inhaler is discarded, a consumer could purchase the securing device and use it with a new inhaler.

It is an object of this invention to provide a holder and securing device for use with a medical inhaler that can be firmly and securely attached to any garment worn by the user so that the inhaler is always available.

It is yet another object of this invention to provide an inhaler encasement and fastener that allows the inhaler encasement to be readily attached to any inhaler body because the encasement is adjustable in size.

In accordance with these and other objects which will become apparent hereinafter, the instant invention will now be described with particular reference to the accompanying drawings.

### BRIEF DESCRIPTION OF DRAWINGS

FIG. 1 shows a perspective exploded view of the invention.

FIG. 2 shows a perspective view of the invention.

FIG. 3 shows an exploded view of an alternate embodiment of the invention.

FIG. 4 shows a perspective view of the invention.

FIG. 5 shows a side elevational view of the invention which is an alternate embodiment.

### DETAILED DESCRIPTION

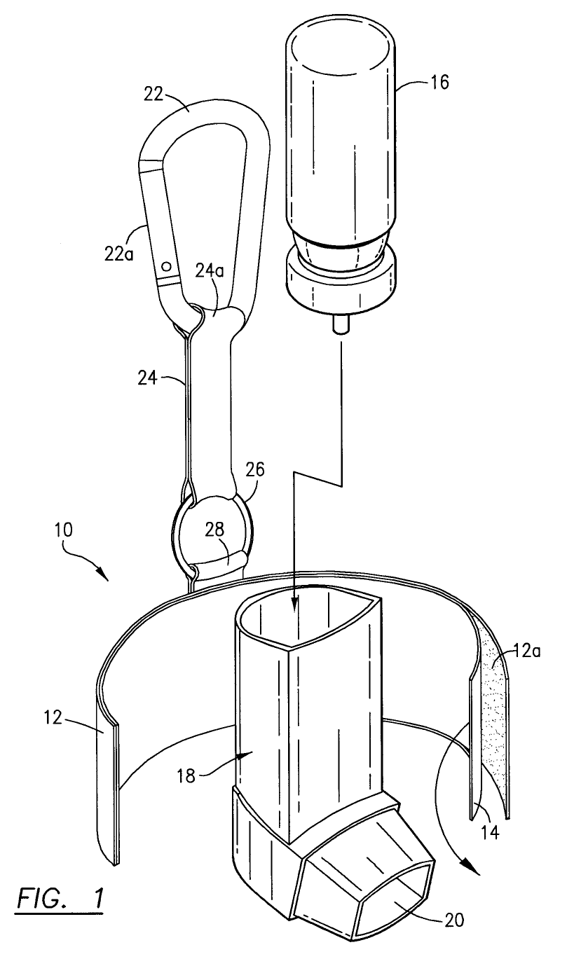

Referring now to the drawings and, in particular, FIG. 1, the present invention is shown generally at 10 comprised of a rectangular, flexible strip of plastic or durable material 12 that includes an adhesive surface 12a and an adhesive protective covering which could be paper 14. The length of the encasing strip 12 is sized so that the strip can extend around an inhaler body 18 which has an opening for the user to spray inhalant into the user's mouth 20 and a container and dispenser 16 that contains the medication. In the conventional operation, a container 16 is depressed which causes a nozzle to expel medicant out opening 20. The present invention encasement strip 12 is attached by the adhesive 12a once the paper 14 is removed so that strip 12 is firmly affixed to the inhaler rigid body 18. The present invention includes a carabiner clip fastener 22 that includes a moveable arm 22a hinged thereto that is attached to a tether strap 24 made of a flexible strong material that include a loop passage 24a permanently attaching the clip fastener 22 thereto. The tether strap 24 is attached to a D ring 26 through a loop 28 at one end. The D ring is permanently attached to the flexible encasement 12 through a loop passage 28 by a suitable fabric or plastic.

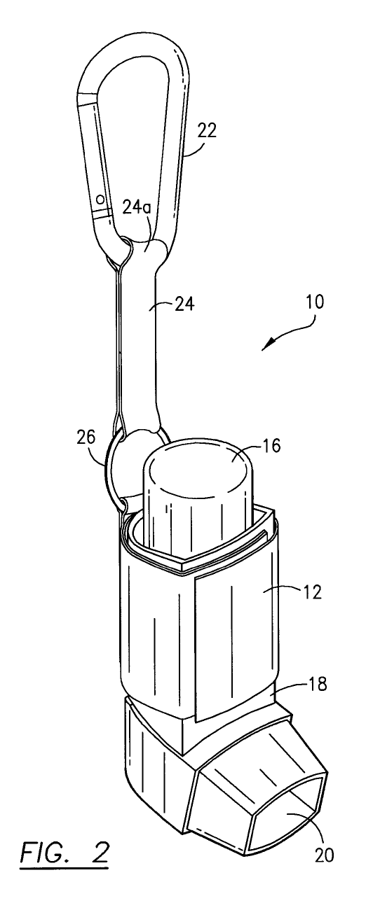

FIG. 2 shows the present invention 10 firmly attached to the inhaler outer body 18 by the adhesive shown in FIG. 1 at 12a. Thus, the encasement 12 cannot be removed since it is glued all the way around the inhaler body 18. To operate the invention, the carabiner clip fastener 22 can be fastened around any belt loop or through any type of aperture in clothing and secured thereto or around clothing straps which could be dress straps or the like. In this configuration, it would be difficult for the inhaler to be removed from the garment.

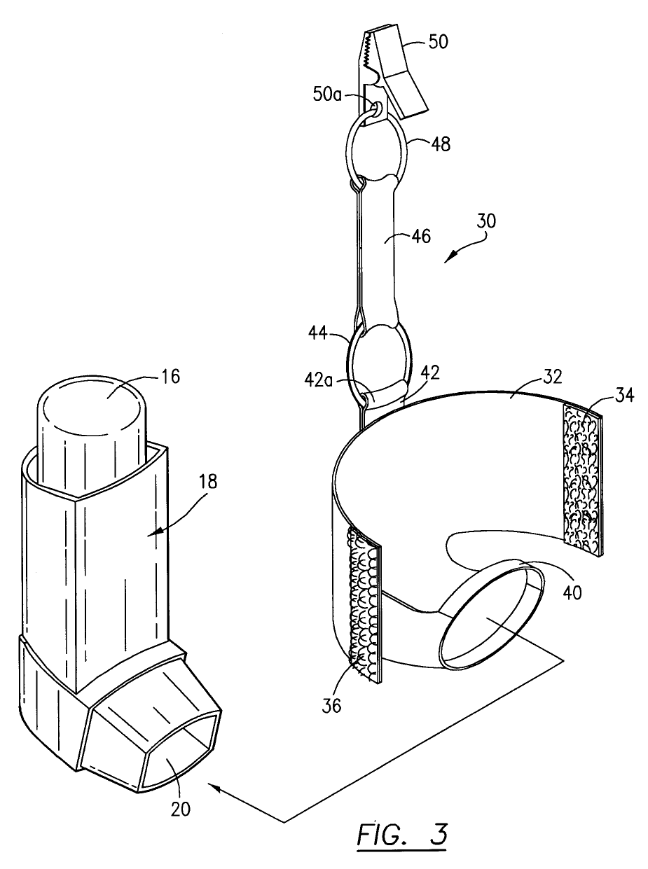

Referring now to FIG. 3, a second embodiment of the invention is shown in which the flexible encasement is a flexible strip 32 that has hook and pile fasteners 34 and 36 at each end which is also sized to fit around the inhaler body 18 in a firm manner. In this embodiment, the encasement 32 can be removed and could be used with other inhalers as the inhaler is expended. The device would also include a loop 40 that fits around the opening 20 on the inhaler body 18 for firmly securing the entire encasement 32 to the inhaler body. Again, attached to the encasement is a loop fastener 42a that retains permanently a D ring 44 which is metal and is attached to the tether strap 46 which could fabric, plastic or leather for permanently holding the D ring 44 to another holder ring 48 attached to alligator clip 50. The alligator clip has an aperture 50a that allows it to be permanently secured to the metal ring 48 which is rigid. Using the alligator clip 50, the device can virtually be attached to any garment. Using the alligator clip 50 which is the preferred embodiment for the invention, users need not fear losing or misplacing their inhalers because they can be attached even to undergarments, thin garments, a bathing suit and literally worn any where. The jaws of the alligator clip 50 are spring loaded closed.

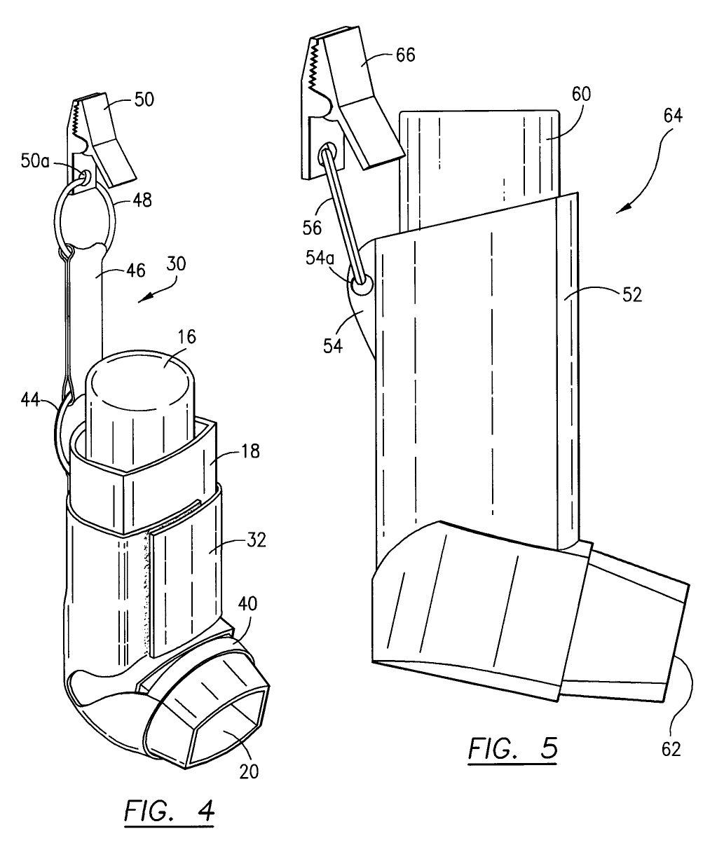

FIG. 4 shows the embodiment shown in FIG. 3 firmly attached around the inhaler body 18. Note that loop 40 which is part of the encasement 32 is attached around the opening 20 of the inhaler body.

Referring now to FIG. 5, an alternate embodiment is shown in which the inhaler body itself has a rigid lip 54 or protrusion that has an aperture 54a that is molded as part of the entire body 52 of the inhaler 64. The mouth opening 62 is for spraying the medicant from container 60 mounted inside the rigid body 52. Attached through the protrusion 54 aperture 54a is a ring fastener 56 which attached to the alligator clip 66. In this embodiment shown in FIG. 5, there is no tether strap but a securing ring 56 to which the alligator clip 66 is attached directly thereto. Again, using the alligator clip 66, there is no danger of losing the inhaler 64 because the clip 66 is firmly securable to any article of clothing at all times to give the user security that the inhaler is with the person of the user.

In summary, using the present invention, a person with asthma or other debilitating disease that must have an inhaling medicine on their person at all times in case of an emergency can feel secure that the device will always be with them because of the attachment of using the present invention.

The instant invention has been shown and described herein in what is considered to be the most practical and preferred embodiment. It is recognized, however, that departures may be made therefrom within the scope of the invention and that obvious modifications will occur to a person skilled in the art.

1. A device for securely fastening an inhaler for dispensing medication to a garment comprising:
an inhaler encasement that includes a flexible, rectangular body sized in length to encompass with a slight overlap an inhaler body;
an adhesive disposed on one side of said encasement body;
protective sheet covering said adhesive prior to use;
a connector connected at one end to said encasement body; and
a spring loaded clip with jaws connected to said connector for attaching said spring loaded clip to a garment, whereby said protective sheet can be removed from said adhesive and said encasement firmly attached to an inhaler body.

2. A device for firmly attaching an inhaler to a garment comprising:
a flexible, rectangular, thin sheet made of a durable material sized in length to fit around an inhaler body;
a hook fastener attached to one end of said flexible sheet on one side and a pile fastener connected to said flexible sheet at the opposite end on the opposite side such that the hook and pile fasteners can be connected together and encompass an inhaler for securing the flexible sheet to an inhaler body;
a fastening loop sized in diameter to fit around a mouth receiving outlet of an inhaler and attached to said flexible sheet for holding said flexible sheet securely to said inhaler;
a connector attached to said flexible sheet; and
a spring loaded clip with jaws attached to said connector whereby said encasement sheet and connector once affixed to an inhaler can be securely attached to a garment.

3. An inhaler for dispensing medication comprising:
an inhaler body that includes a cylindrical passage and an outlet passage for dispensing medication;
a medication container that include a nozzle and spray outlet mountable within said inhaler body;
a connector permanently attached to said inhaler body; and
a spring loaded clip with jaws connected to said connector whereby said inhaler body can be attached by the spring loaded clip to a garment securely.

4. A device as in claim 1, including:
said connector including a strap that is flexible and is used to tether the spring loaded clip to said encasement.

5. A device as in claim 1, wherein:
said spring loaded clip is replaced with a D shaped clip.

6. A device as in claim 1, including:
said spring loaded clip with jaws being an alligator clip.

7. A device as in claim 3, including:
said connector including a strap that is flexible and is used to tether the spring loaded clip to said encasement.

8. A device as in claim 3, wherein:
said spring loaded clip is replaced with a D shaped clip.

9. A device as in claim 3, including:
said spring loaded clip with jaws being an alligator clip.
